(19) United States
(12) Patent Application Publication
(10) Pub. No.: US 2023/0048768 A1
(43) Pub. Date: Feb. 16, 2023

(54) Method of Recovering Nitrogen and Sulfur Resources Through Anaerobic Fermentation

(71) Applicant: SK Innovation Co., Ltd., Seoul (KR)

(72) Inventors: Yeon Hwa La, Daejeon (KR); Tae Wan Kim, Daejeon (KR)

(21) Appl. No.: 17/879,015

(22) Filed: Aug. 2, 2022

(30) Foreign Application Priority Data
Aug. 3, 2021 (KR) 10-2021-0101931

(51) Int. Cl. C12P 7/42; C12N 1/20

(52) U.S. Cl. CPC C12P 7/42; C12N 1/205; C12R 2001/01

(57) ABSTRACT

A method of preparing ammonium sulfate includes feeding biosulfur and ammonia produced during anaerobic fermentation into a sulfur-oxidizing microbial reactor to cause sulfur-oxidizing microorganisms to produce sulfuric acid through oxidation of the biosulfur and reacting the produced sulfuric acid with ammonia to produce the ammonium sulfate. A culture medium containing the produced ammonium sulfate and microorganisms can be used as fertilizers.

### CROSS-REFERENCE TO RELATED APPLICATION

This application claims priority to Korean Patent Application No. 10-2021-0101931 filed Aug. 3, 2021, the disclosure of which is hereby incorporated by reference in its entirety.

### BACKGROUND OF THE INVENTION

### Field of the Invention

The present invention relates to a method of recovering nitrogen and sulfur resources through anaerobic fermentation and more particularly, to a method of preparing ammonium sulfate including feeding biosulfur and ammonia produced during anaerobic fermentation into a sulfur-oxidizing microbial reactor so the sulfur-oxidizing microorganisms oxidize the biosulfur to produce sulfuric acid and reacting the produced sulfuric acid with ammonia to produce the ammonium sulfate.

### Description of Related Art

A methane production process or landfill facility process using organic waste is carried out through anaerobic fermentation of microorganisms. During this anaerobic fermentation, large amounts of reduced sulfur such as hydrogen sulfide and ammonia are produced. These substances are toxic and thus must be treated below permissible standards using a toxic emission prevention facility, before being discharged.

There are two desulfurization methods for hydrogen sulfide: dry desulfurization using iron oxide or activated carbon and wet desulfurization including scrubbing with an alkali solution. Dry desulfurization causes production of a waste desulfurization agent, thus requiring waste treatment. Wet desulfurization has a drawback of entailing the cost for treating the produced wastewater. Biological desulfurization entails a high treatment facility installation cost, but has an advantage of obtaining biosulfur products useful as resources. Since biosulfur has small particles and is produced in a liquid form, it is capable of replacing conventional chemical fertilizers and is useful as organic agricultural materials for pest control and raw materials for pesticides, and thus is considered to have a high potential. Accordingly, a precise separation and purification process is required in order to use biosulfur as a raw material. In addition, all of a biosulfur-containing filtrate generated during conventional hydrogen sulfide pretreatment has been treated as wastewater. However, there is a need for approaches utilizing the biosulfur-containing filtrate generated during treatment of a great amount of hydrogen sulfide in order to reduce the cost of wastewater treatment.

Ammonia is mainly discharged as wastewater, is subjected to nitrification and denitrification using activated sludge and then is removed in the form of N₂. In this process, an additional cost for water treatment is entailed. In particular, ammonia, which is a main raw material for nitrogenous fertilizers, involves a process that emits a large amount of greenhouse gases, so a great deal of research is conducted on alternative strategies. Chemical ammonium sulfate is obtained by producing sulfuric acid from molten sulfur produced at an oil refinery and reacting the sulfuric acid with ammonia produced through the Haber-Bosch process. In this process, the flow of materials obtained from nature and discharged back to nature is complicated and large amounts of greenhouse gases are emitted.

Therefore, there is a need for an innovative fertilizer production method that reduces emission of greenhouse gas.

Therefore, in order to solve the above problem, the present inventors have found that, when hydrogen sulfide produced during anaerobic fermentation or reduced sulfur including biosulfur obtained through desulfurization and ammonia are injected into a sulfur-oxidizing microbial reactor, the sulfur-oxidizing microorganisms oxidize the hydrogen sulfide or biosulfur to produce sulfuric acid, the produced sulfuric acid reacts with ammonia to produce ammonium sulfate and a culture medium containing the produced ammonium sulfate and the microorganisms can be used as fertilizers, and the effect of resource circulation to return nitrogen and sulfur resources obtained from nature back to nature can be maximized. Based on this finding, the present invention was completed.

### RELATED ART DOCUMENT

### Patent Document

(Patent Document 1) EP Patent Laid-open Publication No. 2629606 A2

### Patent Document

(Non-patent Document 1) Young-min Kim, Hyo-soon Song, Hyo-seong Ahn, and Seung-kyu Chun, “Application of the Microbial Process for Hydrogen Sulfide Removal and Bio-Sulfur Production from Landfill Gas”, New & Renewable Energy 2020. 3 Vol. 16, No. 1

### SUMMARY OF THE INVENTION

It is an object of the present invention to provide a method of recovering nitrogen and sulfur resources through anaerobic fermentation by producing an ammonium sulfate-containing fertilizer or microbial product that is capable of maximizing a resource circulation effect of returning nitrogen and sulfur resources obtained from nature back to nature through production of fertilizers using, as raw materials, toxic substances to be treated.

In order to accomplish the above object, the present invention provides a method of preparing ammonium sulfate comprising: passing biosulfur and ammonia through a sulfur-oxidizing microbial reactor to produce sulfuric acid by oxidation of the biosulfur by sulfur-oxidizing microorganisms; and reacting the produced sulfuric acid with ammonia to produce the ammonium sulfate.

### BRIEF DESCRIPTION OF THE DRAWING

The above and other objects, features, and other advantages of the present invention will be more clearly understood from the following detailed description taken in conjunction with the accompanying drawings, in which:

FIG. 1 is a brief schematic diagram illustrating an ammonium sulfate production process according to an embodiment of the present invention.

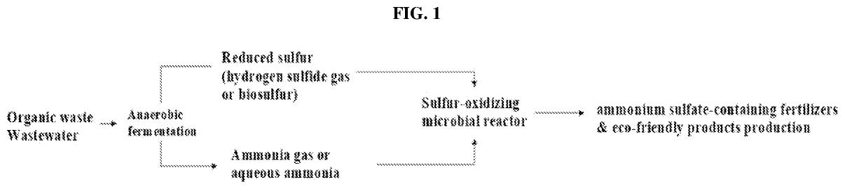

### DESCRIPTION OF THE INVENTION

Unless otherwise defined, all technical and scientific terms used in the present specification have the same meanings as commonly understood by those skilled in the art to which the present disclosure pertains. In general, the nomenclature used in the present specification is well known and commonly used in the art.

Unless defined otherwise, all technical and scientific terms used herein have the same meanings as appreciated by those skilled in the field to which the present invention pertains. In general, the nomenclature used herein is well-known in the art and is ordinarily used.

The present invention is based on the finding that when biosulfur and ammonia produced during anaerobic fermentation are injected into a sulfur-oxidizing microbial reactor, sulfur-oxidizing microorganisms oxidize biosulfur to produce sulfuric acid, the produced sulfuric acid reacts with ammonia to produce ammonium sulfate, and a culture solution containing the produced ammonium sulfate and microorganisms has an effect of being usable as fertilizers.

Accordingly, in one aspect, the present invention is directed to a method of preparing ammonium sulfate comprising: passing biosulfur and ammonia through a sulfur-oxidizing microbial reactor to produce sulfuric acid by oxidation of the biosulfur by sulfur-oxidizing microorganisms; and reacting the produced sulfuric acid with ammonia to produce the ammonium sulfate.

As used herein, the term “sulfur-oxidizing microbial reactor” or a “microbial reactor for producing sulfuric acid” refers to a reactor in which sulfur-oxidizing microorganisms are cultured in a sulfur-containing medium.

The term “microbial reactor” refers to a fermenter including a configuration in which at least one vessel and/or tower or pipe is arranged and may be any appropriate bioreactor including a continuous stirred tank reactor (CSTR), an immobilized cell reactor (ICR), a gas lift reactor, a bubble column reactor (BCR), a membrane reactor such as hollow fiber membrane bioreactor (HFMBR), or a trickle bed reactor (TBR).

The method according to the present invention may be carried out by a fed-batch method in which a substrate is fed to the bioreactor at a specified time and the product remains in the bioreactor until the reaction time elapses, or by a perfusion, continuous, batch, or draw and fill method in which substrates are continuously fed to the bioreactor and by-products are continuously removed while the substrates are damaged.

In addition, the sulfur-oxidizing microorganisms may grow using reduced sulfur as an energy source and carbon dioxide as a carbon source.

In the present invention, the sulfur-oxidizing microorganisms may include at least one selected from the group consisting of bacteria including Acidithiobacillus, Thiobacillus, Thiosphaera, Thermothrix, Beggiatoa, Thioploca, Thiodendron, Thiobacterium, Macromonas, Achromatium, Thiospira, Thioalkalimicrobium, and Thioalkalispira, and Archaea including Sulfolobus and Acidianus.

More specific examples of the sulfur-oxidizing microorganisms according to the present invention are as follows.

A. Acidithiobacillus: Acidithiobacillus Thiooxidans, Acidithiobacillus albertensis, Acidithiobacillus caldus, Acidithiobacillus cuprithermicus, Acidithiobacillus ferridurans, Acidithiobacillus ferrivorans or Acidithiobacillus ferrooxidans

B. Thiobacillus: Thiobacillus denitrificans

C. Thiosphaera: Thiosphaera pantotropha

D. Thermothrix: Thermothrix thiopara

E. Beggiatoa: Beggiatoa alba, Beggiatoa leptomitoformis

F. Thioploca: Thioploca araucae, Thioploca chileae, Thioploca ingrica, Thioploca schmidlei

G. Thiodendron: Thiodendron latens

H. Thiobacterium: Thiobacterium bovistum

I. Macromonas: Macromonas bipunctata

J. Achromatium: Achromatium oxaliferum

K. Thiospira: Thiospira winogradskyi

L. Thioalkalimicrobium: Thioalkalimirobium aerophilum, Thioalkalimicrobium cyclicum

M. Thioalkalispira: Thioalkalispira microaerophila

N. Sulfolobus: Sulfolobus solfataricus

O. Acidianus: Acidianus infernus

In the present invention, the biosulfur may be produced in a desulfurization facility. For example, the biosulfur may be produced in large amounts during anaerobic fermentation in stalls including hog houses and poultry houses, sewage and wastewater treatment plants, manure treatment plants, landfills, food treatment plants, and waste treatment plants.

### EXAMPLE

Hereinafter, the present invention will be described in more detail with reference to the following examples. However, it will be obvious to those skilled in the art that the following examples are provided only for illustration of the present invention, and should not be construed as limiting the scope of the present invention.

### Example 1: Pre-Culture of Sulfur-Oxidizing Microorganisms

50 ml of a medium containing 1 g/L of (NH₄)₂SO₄, 0.5 g/L of MgSO₄.7H₂O, 250 mg/L of CaCl₂.2H₂O, 3 g/L of KH₂PO₄, 10 mg/L of FeSO₄.7H₂O, and 10 g/L of biosulfur was placed in a 100 ml flask and was inoculated with 1 ml of a sulfur-oxidizing microorganism (Acidithiobacillus thiooxidans AZ11, accession number KCTC 8929P). The microorganism was cultured in a shaking incubator at a culture temperature of 30° C. and 150 rpm for 7 days and then was used for main culture inoculation.

### Example 2: Confirmation of Production of Ammonium Sulfate

1,500 ml of a medium containing 1 g/L of (NH₄)₂SO₄, 0.5 g/L of MgSO₄.7H₂O, 250 mg/L of CaCl₂.2H₂O, 3 g/L of KH₂PO₄, 10 mg/L of FeSO₄.7H₂O, and 30 g/L of biosulfur was placed in a 3 L reactor and was inoculated with 50 ml of the sulfur-oxidizing microorganism pre-culture. After culturing at a culture temperature of 37° C. for one day, the pH started to decrease due to sulfuric acid. At this time, aqueous ammonia was continuously injected for pH control so that ammonium sulfate was produced in the reactor. After culturing in a batch mode for 4 days, ammonium sulfate was obtained at a concentration of 91.8 g/L and a sulfur-oxidizing microorganism was obtained at a concentration of 3.1*10¹⁰ cells/ml, as shown in Table 1.

TABLE 1

| | | Sulfur-oxidizing |
|---|---|---|
| Time (day) | Ammonium sulfate (g/L) | microorganism (cells/ml) |
| 0.0 | 2.0 | 1.5E+08 |
| 1.0 | 16.9 | 3.2E+09 |
| 1.3 | 35.1 | 1.3E+10 |
| 1.9 | 67.4 | 2.7E+10 |
| 2.4 | 78.0 | 4.9E+10 |
| 3.0 | 86.0 | 4.2E+10 |
| 3.4 | 87.0 | 3.8E+10 |
| 4.0 | 91.8 | 3.1E+10 |

[Deposit Information]

Name of deposit institution: Korea Research Institute of Bioscience and Biotechnology

Accession number: KCTC8929P

Deposit date: 19990205

### INDUSTRIAL APPLICABILITY

The method of preparing ammonium sulfate according to the present invention includes allowing biosulfur and ammonia to pass through a sulfur-oxidizing microbial reactor to cause the sulfur-oxidizing microorganisms to produce sulfuric acid through oxidation of the biosulfur and reacting the produced sulfuric acid with ammonia to produce the ammonium sulfate. A culture medium containing the produced ammonium sulfate and the microorganisms can be used as fertilizers and thus the effect of resource circulation to return nitrogen and sulfur resources obtained from nature back to nature can be maximized.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

1. A method of preparing ammonium sulfate comprising:
passing biosulfur and ammonia through a sulfur-oxidizing microbial reactor to produce sulfuric acid by oxidation of the biosulfur by sulfur-oxidizing microorganisms; and
reacting the produced sulfuric acid with ammonia to produce the ammonium sulfate.

2. The method of preparing ammonium sulfate of claim 1, wherein the sulfur-oxidizing microorganisms are at least one selected from the group consisting of Acidithiobacillus, Thiobacillus, Thiosphaera, Thermothrix, Beggiatoa, Thioploca, Thiodendron, Thiobacterium, Macromonas, Achromatium, Thiospira, Thioalkalimicrobium, Thioalkalispira, Sulfolobus and Acidianus.

3. The method of preparing ammonium sulfate of claim 1, wherein the biosulfur is produced in a desulfurization facility.
